(19) United States
(12) Patent Application Publication
(10) Pub. No.: US 2026/0272776 A1
(43) Pub. Date: Sep. 17, 2026

(54) ACUPUNCTURE APPARATUS FOR BLIND METHOD

(71) Applicant: Anhui University of Chinese Medicine, Hefei City (CN)

(72) Inventors: Shengbing Wu, Hefei City (CN); Meiqi Zhou, Hefei City (CN); Xuetao Zhang, Hefei City (CN); Shuai Cui, Hefei City (CN); Haiyan Zuo, Hefei City (CN); Kun Wang, Hefei City (CN); Xin Wu, Hefei City (CN); Nenggui Xu, Hefei City (CN)

(73) Assignee: Anhui University of Chinese Medicine, Hefei City (CN)

(21) Appl. No.: 19/442,046

(22) Filed: Jan. 7, 2026

(30) Foreign Application Priority Data
Mar. 14, 2025 (CN) 202510302227.X

(51) Int. Cl. A61H 39/08

(52) U.S. Cl. CPC A61H 39/083; A61H 39/086

(57) ABSTRACT

The present disclosure belongs to the technical field of acupuncture apparatuses and discloses an acupuncture apparatus for a blind method. The acupuncture apparatus includes a treatment needle kit. The treatment needle kit includes a first needle tube and a first descending control component as well as a first depth adjustment component, a first descending component and a treatment needle which are arranged in the first needle tube in sequence from top to bottom. The first descending component drives the treatment needle to move downwards in the first needle tube. The first descending control component is configured to adjust a position of the first descending component in the first needle tube so as to control a descending distance of the treatment needle. The treatment needle kit of the present disclosure can control the descending distance of the treatment needle, so as to achieve controlling over a depth of acupuncture.

### TECHNICAL FIELD

The present disclosure belongs to the technical field of acupuncture apparatuses, and particularly relates to an acupuncture apparatus for a blind method.

### BACKGROUND OF THE INVENTION

In medical study and clinical practice, a double blind experiment, as a strict clinical and trial study method, is widely applied to evaluation of effects of medicine, therapy or intervening measures. A core characteristic of the double blind experiment lies in that both an experimenter and a subject are unaware of which subjects are subjected to a real treatment (experimental group) or placebo (control group), so that an influence of a subjective prejudice and a placebo effect on a clinical study result is reduced to the maximum degree.

However, in the double blind experiment, implementing a “blind method” for acupuncture is a challenge. A conventional acupuncture treatment depends on a needling instrument pricking into a specific acupuncture point of a human body and generating a treatment effect through a manipulation. During the process, both the experimenter and the subject can directly feel presence and stimulation of the needling instrument, so a real double blind is hardly achieved.

An existing treatment acupuncture apparatus has a problem of uncontrollable acupuncture depth, an existing placebo acupuncture apparatus has problems of low concealment, a large difference of acupuncture feeling and the like, and these differences may cause a change in a psychological expectation, reaction and subjective evaluation of an experiment subject, so accuracy of a study result is affected.

Thus, the development of a treatment acupuncture apparatus that can achieve an adjustable and controllable acupuncture depth for better simulating a real acupuncture feeling, achieving a placebo acupuncture apparatus with a higher degree of concealment and being capable of setting an acupuncture depth have a significance on improvement of accuracy of acupuncture treatment evaluation in the double blind experiment.

### SUMMARY OF THE INVENTION

For the above problem, the present disclosure provides an acupuncture apparatus for a blind method. The following technical solution is adopted.

The acupuncture apparatus for the blind method includes a treatment needle kit. The treatment needle kit includes a first needle tube and a first descending control component as well as a first depth adjustment component, a first descending component and a treatment needle which are arranged in the first needle tube in sequence from top to bottom;

where the first depth adjustment component is connected with the first descending component, a top of the treatment needle is fixedly connected with the first descending component, and the first descending component drives the treatment needle to move downwards in the first needle tube; and the first descending control component is arranged in the first needle tube and located below the first descending component, and the first descending control component is configured to adjust a position of the first descending component in the first needle tube so as to control a descending distance of the treatment needle.

Further, both a top and a bottom of the first needle tube are open, a snap-in opening which penetrates through a tube wall is formed in a position, at the first descending component, of the first needle tube, an outer side wall of the first needle tube is provided with an elastic push part at the snap-in opening, an upper end of the elastic push part is fixedly connected with the outer side wall of the first needle tube, and a first protrusion block matching the snap-in opening is arranged on an inner side of a lower end of the elastic push part.

Further, the first descending component includes a first reset tube and a first connecting cylinder;

where the first reset tube is arranged in the first needle tube and located below the first depth adjustment component, and a first cavity is formed between the first reset tube and the first needle tube; the first reset tube is arranged outside the first connecting cylinder in a sleeving mode, an outer wall of the first connecting cylinder is provided with limiting rings at two ends of the first reset tube, and an outer diameter of each limiting ring is greater than an outer diameter of the first reset tube; and

a first groove is formed in a position, close to the snap-in opening, of a tube wall of a top of the first reset tube, a first buckle is arranged in the first groove, the first buckle is in a strip shape, a top of the first buckle is fixedly connected with a wall surface of a top of the first groove, a second protrusion block matching the snap-in opening is arranged on an outer side surface of a bottom of the first buckle, the second protrusion block, when unstressed, penetrates through the first cavity to be snapped into the snap-in opening, and the second protrusion block slides in the first cavity.

Further, a strip-shaped reset groove is formed in a lower portion of a tube wall of the first needle tube, the reset groove extends in an axial direction of the first needle tube, a first reset rod is arranged on the first reset tube, the first reset rod penetrates through the first cavity and the reset groove, one end of the first reset rod is fixedly connected with an outer side wall of a bottom of the first reset tube, the other end of the first reset rod is located outside the first needle tube, and the other end of the first reset rod is provided with a spherical handle.

Further, the first needle tube is provided with a color recognizing port in an upper portion of the elastic push part, and the first depth adjustment component includes a first color recognizing part and a first spring;

where the first color recognizing part is arranged at a top opening of the first needle tube, the first color recognizing part includes a first color recognizing body and a first spring sleeve, a bottom of the first color recognizing body is rotationally connected with the top opening of the first needle tube, and a top of the first color recognizing body is located outside the first needle tube; and a plurality of color regions are arranged in a position, having the same height as the color recognizing port of the first needle tube, of an outer side surface of the first color recognizing body in a circumferential direction, and colors between the plurality of color regions are different.

Further, the first depth adjustment component further includes a first internal gear, and the first descending component further includes a first external gear;

a blind hole is formed in a middle of the bottom of the first color recognizing body in an axial direction, a top of the first spring sleeve is in interference fit with the blind hole, the first internal gear is arranged on an outer side of the first spring sleeve in a sleeving mode and located at the bottom of the first color recognizing body, a top of the first connecting cylinder is provided with the first external gear engaged with the first internal gear, a first base plate is fixedly arranged below the limiting ring at a bottom of the first connecting cylinder, and the top of the treatment needle is fixedly connected with a bottom of the first base plate; and

a bottom of the first spring sleeve is located in the first connecting cylinder, the first spring is arranged in the first spring sleeve, a top of the first spring is in contact with a bottom of the blind hole, and a bottom of the first spring is in contact with the first base plate.

Further, the first descending control component includes an arc-shaped descending control part, an outer wall surface of the arc-shaped descending control part is attached to an inner wall surface of the first needle tube, the arc-shaped descending control part is provided with a plurality of step surfaces having different heights in a circumferential direction, a position of each step surface corresponds to a position of one color region on the first color recognizing part, an arc-shaped first stop block is arranged on an outer side surface of the first base plate, an arc length of a cross section of the first stop block is equal to an arc length of each step surface, and each step surface completely coincides with the first stop block in a vertical direction.

Further, the first depth adjustment component includes a first plunger and a fourth spring;

where the first plunger is in a “T” shape and includes a third cylindrical portion and a fourth cylindrical portion which are connected in sequence from top to bottom, an outer diameter of the third cylindrical portion is greater than an outer diameter of the fourth cylindrical portion, the third cylindrical portion is rotationally connected with a top opening of the first needle tube, the fourth cylindrical portion is located in the first needle tube, a second cavity is formed between the fourth cylindrical portion and the first needle tube, and the fourth spring is located in the second cavity and arranged on the fourth cylindrical portion in a sleeving mode.

Further, the first descending component includes a first descending tube;

where the first descending tube is arranged on an outer side of the fourth spring in a sleeving mode and located between the first needle tube and the fourth spring, a second stop block is arranged at a bottom of the first descending tube, and a bottom of the second stop block is fixedly connected with the treatment needle; and a second buckle matching the snap-in opening of the first needle tube is arranged on an outer side surface of the first descending tube.

Further, the first descending component further includes a second reset rod, the second reset rod penetrates through a reset groove of a lower portion of a tube wall of the first needle tube, one end of the second reset rod is fixedly connected with the second stop block, the other end of the second reset rod is located outside the first needle tube, and the other end of the second reset rod is provided with a spherical handle.

Further, the first descending control component includes a plurality of descending control buttons which are vertically distributed on the first needle tube;

where each descending control button includes a press plate, a motion bar, two clamping blocks, a locking block and a stop bar, a third round hole and a fourth round hole which communicate with each other are formed in sequence in a position, at each descending control button, of a tube wall of the first needle tube from outside to inside, and an inner diameter of the fourth round hole is greater than an inner diameter of the third round hole;

the two clamping blocks are oppositely arranged in the fourth round hole, one ends, close to the third round hole, of the two clamping blocks are fixedly connected with a side wall of the fourth round hole, and a third cavity is formed between the two clamping blocks;

the press plate is arranged outside the first needle tube, the hemispherical locking block is arranged in the third cavity, one end of the motion bar is fixedly connected with the press plate, the other end of the motion bar penetrates through the third round hole to be fixedly connected with the locking block, the motion bar is in sliding connection with the third round hole, the stop bar is arranged in the fourth round hole and located at one ends, away from the third round hole, of the two clamping blocks, and the locking block is fixedly connected with the stop bar; and

the third cavity includes a cylindrical segment and an arc-shaped segment which communicate with each other from outside to inside, and an arc surface of the arc-shaped segment and an arc surface of the locking block are tangential to form clamping for the locking block.

Further, the acupuncture apparatus for the blind method further includes a placebo needle kit having the consistent shape as the treatment needle kit, the placebo needle kit includes a second needle tube and a second descending control component as well as a second depth adjustment component, a second descending component and a placebo needle which are arranged in the second needle tube in sequence from top to bottom;

where the second depth adjustment component is connected with the second descending component, the placebo needle is in sliding connection with the second descending component, the second descending control component is arranged in the second needle tube and located below the second descending component, the second descending control component is configured to adjust a position of the second descending component in the second needle tube, and the second descending component is configured to control a descending distance of the placebo needle to be a set value when moving downwards in the second needle tube.

Further, the second depth adjustment component includes a second color recognizing part and a third spring;

the second color recognizing part is arranged at a top opening of the second needle tube and includes a second color recognizing body and a second spring sleeve, a bottom of the second color recognizing body is rotationally connected with the top opening of the second needle tube, and a top of the second color recognizing body is located outside the second needle tube;

a first round hole and a second round hole which communicate with each other are formed in a middle of the second color recognizing body in sequence from top to bottom in an axial direction, and an inner diameter of the second round hole is greater than an inner diameter of the first round hole; and a top of the second spring sleeve is in interference fit with the second round hole.

Further, the second descending component includes a second reset tube, a second connecting cylinder, an inner spring sleeve, a first acupuncture fixing rod and a second spring;

where a second base plate is fixedly arranged below the limiting ring at a bottom of the second connecting cylinder, and the bottom of the second color recognizing body is in transmission connection with a top of the second connecting cylinder;

a bottom of the second spring sleeve is located in the second connecting cylinder, a top of the inner spring sleeve is fixedly connected with a bottom of the second round hole, the second spring is arranged outside the inner spring sleeve in a sleeving mode, a top of the second spring is in contact with the bottom of the second round hole, and a bottom of the second spring is in contact with the second base plate;

the first acupuncture fixing rod is located in the inner spring sleeve, a top of the first acupuncture fixing rod is in sliding connection with the first round hole, and a bottom of the first acupuncture fixing rod penetrates through the second base plate to be fixedly connected with the placebo needle; and

the first acupuncture fixing rod is provided with a first stop ring and a second stop ring, the first stop ring is located in the inner spring sleeve, the second stop ring is located below the inner spring sleeve, a first inner flange is arranged at a bottom of the inner spring sleeve and located between the first stop ring and the second stop ring, a second groove matching the second stop ring is formed in a connection position of the second base plate and the second connecting cylinder, the third spring is arranged on the first acupuncture fixing rod in a sleeving mode, a top of the third spring is in contact with a top of the second round hole, and a bottom of the third spring is in contact with the first stop ring.

Further, the second depth adjustment component includes a second plunger and a fifth spring;

where the second plunger is in a “T” shape and includes a fifth cylindrical portion and a sixth cylindrical portion which are connected in sequence from top to bottom, an outer diameter of the fifth cylindrical portion is greater than an outer diameter of the sixth cylindrical portion, the fifth cylindrical portion is rotationally connected with the top opening of the second needle tube, the sixth cylindrical portion is located in the second needle tube, a fourth cavity is formed between the sixth cylindrical portion and the second needle tube, and the fifth spring is located in the fourth cavity and arranged on the sixth cylindrical portion in a sleeving mode.

Further, the second descending component includes a second descending tube, a second acupuncture fixing rod and a sixth spring;

where the second descending tube is arranged on an outer side of the fifth spring in a sleeving mode and located between the second needle tube and the fifth spring, and a third stop block is arranged at a bottom of the second descending tube;

a fifth cavity is formed in the second plunger in an axial direction, the second acupuncture fixing rod is located in the fifth cavity, and a bottom of the second acupuncture fixing rod penetrates through the third stop block to be fixedly connected with the placebo needle; and

the second acupuncture fixing rod is provided with a third stop ring and a fourth stop ring, a second inner flange is arranged at a bottom of the second plunger and located between the third stop ring and the fourth stop ring, the third stop ring is located in the fifth cavity, the fourth stop ring is located below the fifth cavity, the sixth spring is arranged on the second acupuncture fixing rod in a sleeving mode, a top of the sixth spring is in contact with a top of the fifth cavity, and a bottom of the sixth spring is in contact with the third stop ring.

Further, a fixing plate is arranged below bottoms of the first needle tube and the second needle tube, and adhesive tape is detachably arranged at a bottom of the fixing plate.

Further, a first through hole is formed in the fixing plate at a position of the adhesive tape, and a blood-mimicking member is arranged in the first through hole.

Further, acupuncture guide blocks are arranged at bottom openings of the first needle tube and the second needle tube, the first descending control component surrounds an outer side of the acupuncture guide block in the first needle tube, and the second descending control component surrounds an outer side of the acupuncture guide block in the second needle tube; and

a second through hole matching the treatment needle in diameter is formed in the acupuncture guide block in the first needle tube, and a second through hole matching the placebo needle in diameter is formed in the acupuncture guide block in the second needle tube.

Further, the treatment needle kit and the placebo needle kit each include an electroacupuncture joint, each electroacupuncture joint is in an L shape, one end of the electroacupuncture joint of the treatment needle kit is located outside the first needle tube, and the other end of the electroacupuncture joint of the treatment needle kit penetrates through the first needle tube to be fixedly connected with a bottom of the first descending component; and

one end of the electroacupuncture joint of the placebo needle kit is located outside the second needle tube, and the other end of the electroacupuncture joint of the placebo needle kit penetrates through the second needle tube to be fixedly connected with a bottom of the second descending component.

The present disclosure has the following beneficial effects.
1, The treatment needle kit of the present disclosure can control the descending distance of the treatment needle through cooperation between the first descending component, the first depth adjustment component and the first descending control component, so as to achieve controlling over a depth of acupuncture, and depths of the treatment needle pricking into the skin are different according to different treatment purposes during actual use so as to meet different demands.
2, The placebo needle kit of the present disclosure can control the pricking depth of the placebo needle to be the set value through cooperation between the second descending component, the second depth adjustment component and the second descending control component, so that the placebo needle can prick the skin in a small depth and cannot achieve the treatment purpose, only a needle hole is left in the skin, and a patient has the same touch feeling.
3, The treatment needle kit and the placebo needle kit of the present disclosure have completely identical appearance, a user and a subject cannot judge whether the current kit is the treatment needle kit or the placebo needle kit through the appearance and thus cannot know whether they participate in a treatment group or a placebo group, experience and feeling as well as the touch feeling caused by an acupuncture manipulation are the same for both an experimenter and a to-be-treated patient, cognitive bias for an acupuncture and moxibustion effect caused by a psychological effect is greatly reduced, and the double blind experiment is effectively implemented.

Other features and advantages of the present disclosure are to be set forth in the following specification, and part of them are apparent in the specification or understood by implementing the present disclosure. Objectives and other advantages of the present disclosure may be achieved and obtained through a structure indicated in the specification and the accompanying drawings.

### BRIEF DESCRIPTION OF DRAWINGS

For more clearly describing the technical solutions in the embodiments of the present disclosure or in the prior art, accompanying drawings needed in the descriptions of the embodiments or the prior art are briefly introduced below. Apparently, the accompanying drawings in the following description are some embodiments of the present disclosure. Those ordinarily skilled in the art can also obtain other accompanying drawings according to these accompanying drawings without making creative efforts.

FIG. 1 shows a schematic structural sectional view of a treatment needle kit according to Embodiment 1 of the present disclosure.

FIG. 2 shows an axonometrical drawing of a treatment needle kit according to Embodiment 1 of the present disclosure.

FIG. 3 shows an enlarged view of a position A in FIG. 1.

FIG. 4 shows an explosive view of a treatment needle kit according to Embodiment 1 of the present disclosure.

FIG. 5 shows a schematic installation diagram of a first color recognizing body and a first spring sleeve according to Embodiment 1 of the present disclosure.

FIG. 6 shows a schematic installation diagram of a first needle tube and a fixing plate according to Embodiment 1 of the present disclosure.

FIG. 7 shows a schematic structural diagram of a first descending control component according to Embodiment 1 of the present disclosure.

FIG. 8 shows a schematic installation diagram of a first connecting cylinder and a first base plate according to Embodiment 1 of the present disclosure.

FIG. 9 shows a schematic structural sectional view of a placebo needle kit according to Embodiment 1 of the present disclosure.

FIG. 10 shows an enlarged view of a position B in FIG. 9.

FIG. 11 shows an axonometrical drawing of a treatment needle kit according to Embodiment 2 of the present disclosure.

FIG. 12 shows a schematic structural sectional view of a treatment needle kit according to Embodiment 2 of the present disclosure.

FIG. 13 shows a schematic structural diagram of a descending control button according to an embodiment of the present disclosure.

FIG. 14 shows a schematic structural sectional view of a placebo needle kit according to Embodiment 2 of the present disclosure.

In the figures: 1, treatment needle kit; 2, placebo needle kit; 3, first needle tube; 4, first descending component; 5, first depth adjustment component; 6, first descending control component; 7, treatment needle; 8, snap-in opening; 9, elastic push part; 10, first protrusion block; 11, first reset tube; 12, first connecting cylinder; 13, limiting ring; 14, first groove; 15, first buckle; 16, second protrusion block; 17, reset groove; 18, first reset rod; 19, color recognizing port; 20, first color recognizing part; 21, first spring; 22, first color recognizing body; 23, first spring sleeve; 24, first internal gear; 25, first external gear; 26, blind hole; 27, first base plate; 28, needle tube cover; 29, first cylindrical portion; 30, second cylindrical portion; 31, positioning step; 32, limiting step; 33, arc-shaped descending control part; 331, step surface; 34, first stop block; 35, second needle tube; 36, second descending component; 37, second depth adjustment component; 38, second descending control component; 39, placebo needle; 40, second reset tube; 41, second connecting cylinder; 42, inner spring sleeve; 43, first acupuncture fixing rod; 44, second spring; 45, second color recognizing body; 46, third spring; 47, second spring sleeve; 48, first round hole; 49, second round hole; 50, second base plate; 51, second internal gear; 52, second external gear; 53, first stop ring; 54, second stop ring; 55, first inner flange; 56, second groove; 57, first plunger; 58, fourth spring; 59, third cylindrical portion; 60, fourth cylindrical portion; 61, first descending tube; 62, second stop block; 63, second buckle; 64, second reset rod; 65, descending control button; 66, press plate; 67, motion bar; 68, clamping block; 69, locking block; 70, stop bar; 71, third round hole; 72, fourth round hole; 73, cylindrical segment; 74, arc-shaped segment; 75, second plunger; 76, fifth spring; 77, second descending tube; 78, second acupuncture fixing rod; 79, sixth spring; 80, third stop block; 81, third stop ring; 82, fourth stop ring; 83, second inner flange; 84, fixing plate; 85, blood-mimicking member; 86, acupuncture guide block; 87, electroacupuncture joint; and 88, adhesive tape.

### DETAILED DESCRIPTION OF THE INVENTION

For making objectives, technical solutions and advantages of the embodiments of the present disclosure clearer, the technical solutions in the embodiments of the present disclosure are described clearly and completely with reference to the accompanying drawings in the embodiments of the present disclosure. Apparently, the described embodiments are some embodiments but not all embodiments of the present disclosure. All other embodiments obtained by those ordinarily skilled in the art based on the embodiments in the present disclosure without making creative efforts fall within the scope of protection of the present disclosure.

It needs to be noted that terms such as “first” and “second” in the present application are used for distinguishing similar objects but not for describing a specific sequence or precedence order. It is to be understood that data used like this may be interchanged in an appropriate case so as to describe the embodiments of the present application. In the present application, directions or position relationships indicated by terms such as “upper,” “lower,” “left,” “right,” “front,” “back,” “top,” “bottom,” “inner,” “outer,” “in,” “vertical,” “horizontal,” “transverse” and “longitudinal” are based on directions or position relationships shown in the accompanying drawings.

The present disclosure provides an acupuncture apparatus for a blind method and is intended to simulate an acupuncture therapy in a double blind clinical study, so as to help to scientifically evaluate a curative effect of the acupuncture, and achieve a controllable acupuncture depth, and problems in the prior art that a placebo acupuncture apparatus has low concealment, a large difference of acupuncture feeling and inconsistent acupuncture depth are solved.

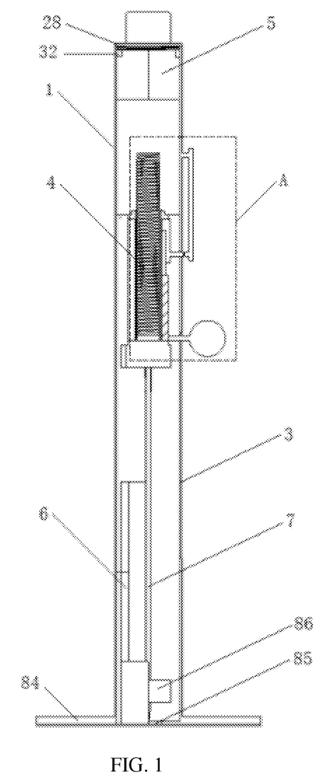

As shown in FIGS. 1 and 9, the acupuncture apparatus for the blind method includes a treatment needle kit 1 and a placebo needle kit 2. The treatment needle kit 1 has a treatment effect, and the placebo needle kit 2 has no treatment effect.

As shown in FIG. 1, the treatment needle kit 1 includes a first needle tube 3, a first descending component 4, a first depth adjustment component 5, a first descending control component 6 and a treatment needle 7. The first depth adjustment component 5, the first descending component 4 and the treatment needle 7 are arranged in the first needle tube 3 in sequence from top to bottom. The first depth adjustment component 5 is connected with the first descending component 4, a top of the treatment needle 7 is fixedly connected with the first descending component 4, and the first descending component 4 drives the treatment needle 7 to move downwards in the first needle tube 3.

The first descending control component 6 is arranged in the first needle tube 3 and located below the first descending component 4, and the first descending control component 6 is configured to adjust a position of the first descending component 4 in the first needle tube 3 so as to control a descending distance of the treatment needle 7. During actual use, according to different treatment purposes, depths of the treatment needle 7 pricking into the skin are different. For meeting different demands, the depths pricking into the skin may be generally set to be 0.5 inch, 1 inch and 1.5 inches respectively.

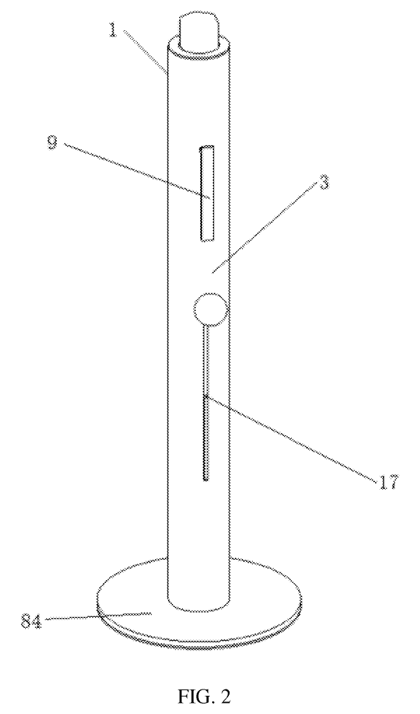

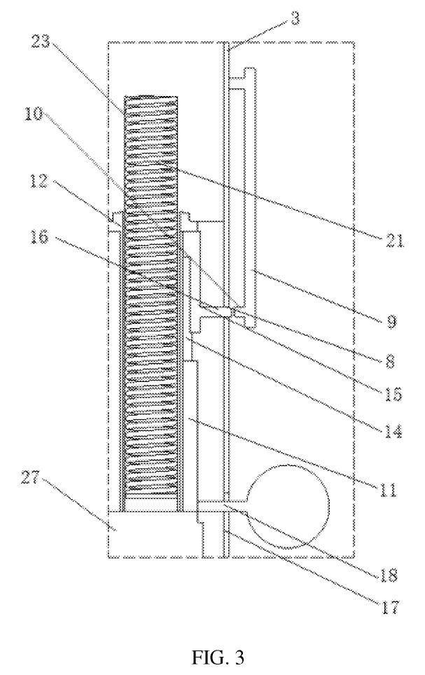

As shown in FIGS. 2 and 3, for example, the first needle tube 3 may be cylindrical, both a top and a bottom of the first needle tube 3 are open, a snap-in opening 8 which penetrates through a tube wall is formed in a position, at the first descending component 4, of the first needle tube 3, an outer side wall of the first needle tube 3 is provided with an elastic push part 9 at the snap-in opening 8, an upper end of the elastic push part 9 is fixedly connected with the outer side wall of the first needle tube 3, a first protrusion block 10 matching the snap-in opening 8 is arranged on an inner side of a lower end of the elastic push part 9, and the first protrusion block 10 is enabled to enter and exit the snap-in opening 8 by pressing or loosening the elastic push part 9.

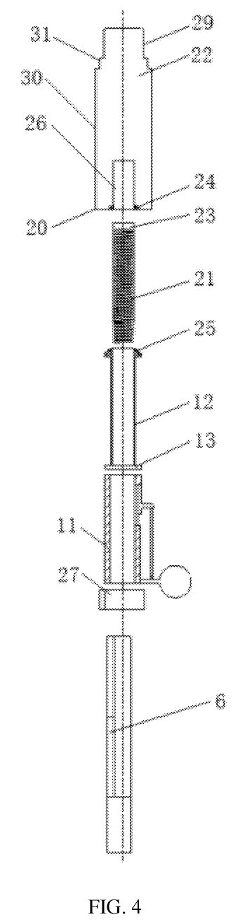

As shown in FIGS. 3 and 4, for example, the first descending component 4 includes a first reset tube 11 and a first connecting cylinder 12. The first reset tube 11 is arranged in the first needle tube 3 and located below the first depth adjustment component 5, the first reset tube 11 and the first needle tube 3 are arranged concentrically, and a first cavity is formed between the first reset tube 11 and the first needle tube 3. The first reset tube 11 is arranged outside the first connecting cylinder 12 in a sleeving mode, an outer wall of the first connecting cylinder 12 is provided with limiting rings 13 at two ends of the first reset tube 11, and an outer diameter of each limiting ring 13 is greater than an outer diameter of the first reset tube 11.

A first groove 14 is formed in a position, close to the snap-in opening 8, of a tube wall of a top of the first reset tube 11, a first buckle 15 is arranged in the first groove 14, the first buckle 15 is in a strip shape, a top of the first buckle 15 is fixedly connected with a wall surface of a top of the first groove 14, a second protrusion block 16 matching the snap-in opening 8 is arranged on an outer side surface of a bottom of the first buckle 15, the second protrusion block 16, when unstressed, penetrates through the first cavity to be snapped into the snap-in opening 8, the first protrusion block 10 at a lower end of the elastic push part 9 pushes the second protrusion block 16 out of the snap-in opening 8 by pressing the elastic push part 9, and the second protrusion block 16 slides in the first cavity.

As shown in FIGS. 2 and 3, a strip-shaped reset groove 17 is formed in a lower portion of a tube wall of the first needle tube 3, the reset groove 17 extends in an axial direction of the first needle tube 3, a first reset rod 18 is arranged on the first reset tube 11, the first reset rod 18 penetrates through the first cavity and the reset groove 17, one end of the first reset rod 18 is fixedly connected with an outer side wall of a bottom of the first reset tube 11, the other end of the first reset rod 18 is located outside the first needle tube 3, and the other end of the first reset rod 18 is provided with a spherical handle, so an operation is convenient.

The first reset tube 11 may complete pulling out of a needle by poking up the reset rod. When idle, the second protrusion block 16 is located in the snap-in opening 8, and after pressing the elastic push part 9, the first descending component 4 moves downwards in the first needle tube 3, so an acupuncture action is completed. When the needle needs to be pulled out, the reset rod may be pushed upwards to snap the second protrusion block 16 into the snap-in opening 8 again.

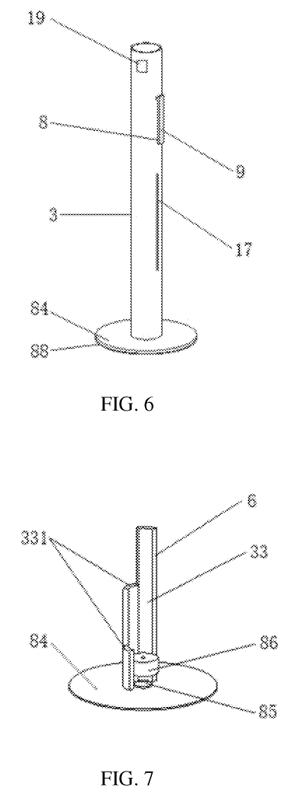

As shown in FIG. 6, the first needle tube 3 is provided with a color recognizing port 19 in an upper portion of the elastic push part 9, and the first depth adjustment component 5 includes a first color recognizing part 20 and a first spring 21. The first color recognizing part 20 is arranged at a top opening of the first needle tube 3.

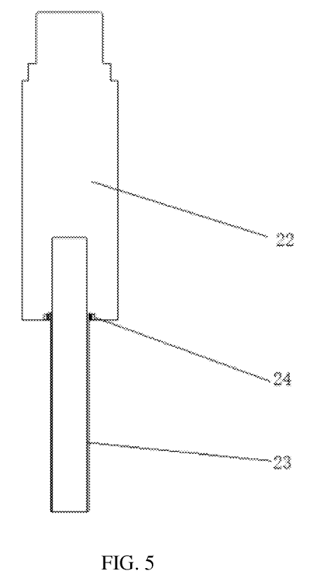

As shown in FIGS. 4 and 5, the first color recognizing part 20 includes a first color recognizing body 22 and a first spring sleeve 23, the first color recognizing body 22 is cylindrical, a bottom of the first color recognizing body 22 is rotationally connected with the top opening of the first needle tube 3, and a top of the first color recognizing body 22 is located outside the first needle tube 3, so a user may rotate the first color recognizing body 22.

A plurality of color regions are arranged in a position, having the same height as the color recognizing port 19 of the first needle tube 3, of an outer side surface of the first color recognizing body 22 in a circumferential direction, and colors between the plurality of color regions are different. The plurality of color regions have the same size as the color recognizing port 19 of the first needle tube 3, each color region is of a small blocky protruding structure, the plurality of color regions horizontally surround the first color recognizing body 22 by one circle, each color region may be caused to rightly fall into the color recognizing port 19 of the first needle tube 3 by rotating the first color recognizing body 22, and different acupuncture depths are selected according to different colors.

As shown in FIGS. 4,5 and 8, the first depth adjustment component 5 further includes a first internal gear 24, and the first descending component 4 further includes a first external gear 25. A blind hole 26 is formed in a middle of the bottom of the first color recognizing body 22 in an axial direction, and a top of the first spring sleeve 23 is in interference fit with the blind hole 26, so that the first spring sleeve 23 is fixed to the blind hole 26. The first internal gear 24 is arranged on an outer side of the first spring sleeve 23 in a sleeving mode and located at the bottom of the first color recognizing body 22, a top of the first connecting cylinder 12 is provided with the first external gear 25 engaged with the first internal gear 24, a disc-shaped first base plate 27 is fixedly arranged below the limiting ring 13 of a bottom of the first connecting cylinder 12, and the top of the treatment needle 7 is fixedly connected with a bottom of the first base plate 27. The first base plate 27, when descending, may drive the treatment needle 7 to descend and extend out of the first needle tube 3, so as to achieve the purpose of acupuncture.

A bottom of the first spring sleeve 23 is located in the first connecting cylinder 12, the first spring 21 is arranged in the first spring sleeve 23, a top of the first spring 21 is in contact with a bottom of the blind hole 26, and a bottom of the first spring 21 is in contact with the first base plate 27.

As shown in FIGS. 1 and 4, for example, a needle tube cover 28 is arranged at the top opening of the first needle tube 3, the needle tube cover 28 limits the first color recognizing body 22 into the first needle tube 3, the first color recognizing body 22 includes a first cylindrical portion 29 and a second cylindrical portion 30 which are connected in sequence from top to bottom, an annular positioning step 31 is arranged at a connection position of the first cylindrical portion 29 and the second cylindrical portion 30, the first cylindrical portion 29 penetrates through the needle tube cover 28, the second cylindrical portion 30 is rotationally connected with the first needle tube 3, the blind hole 26 is formed in a bottom of the second cylindrical portion 30, and an annular limiting step 32 matching the annular positioning step 31 is arranged at a bottom of the needle tube cover 28.

As shown in FIG. 7, for example, the first descending control component 6 includes an arc-shaped descending control part 33, an outer wall surface of the arc-shaped descending control part 33 is attached to an inner wall surface of the first needle tube 3, the arc-shaped descending control part 33 is provided with a plurality of step surfaces 331 having different heights in a circumferential direction, and a position of each step surface 331 corresponds to a position of one color region on the first color recognizing part 20.

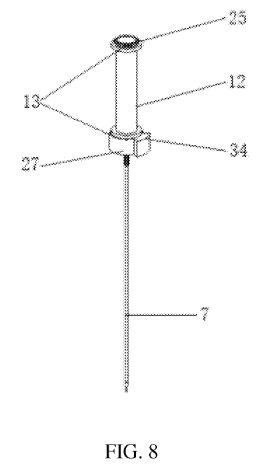

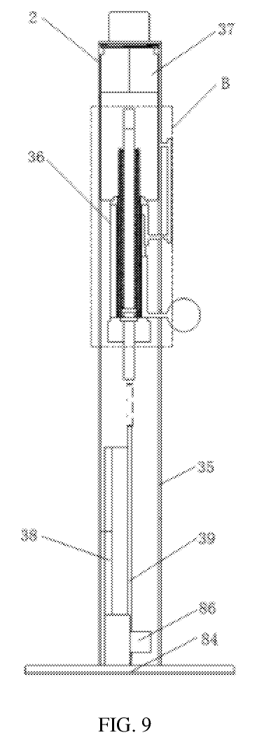

As shown in FIG. 8, an arc-shaped first stop block 34 is arranged on an outer side surface of the first base plate 27, an arc length of a cross section of the arc-shaped first stop block 34 is equal to an arc length of each step surface 331, and each step surface 331 completely coincides with the first stop block 34 in a vertical direction.

In the above technical solution, when idle, the first reset tube 11 is also in a fixed state under an action of the limiting rings 13 at the two ends of the connecting cylinder, and the first spring 21 is in a compressed state. When in use, the first color recognizing part 20 is rotated to align the color region of a required acupuncture depth to the color recognizing port 19. The different color regions represent different descending ranges of the treatment needle 7, namely, different acupuncture depths. For example, red represents that the first stop block 34 may be blocked by a highest step surface 331 of the arc-shaped descending control part 33, thus the treatment needle can descend by only 0.5 inch, namely, the acupuncture depth is also 0.5 inch. Green represents that the treatment needle 7 may descend by one inch, namely, the acupuncture depth is also one inch. Purple represents that the treatment needle 7 may descend by 1.5 inches, namely, the acupuncture depth is also 1.5 inches.

The first color recognizing part 20 is rotated so as to drive the first base plate 27 to rotate under an action of the first external gear 25 and the first internal gear 24, and in this case, the corresponding first stop block 34 may be rightly located above the step surface 331, corresponding to this color, of the arc-shaped descending control part 33. The elastic push part 9 is pressed to separate the second protrusion block 16 from the snap-in opening 8, the first reset tube 11 loses support, and the first connecting cylinder 12 and the first base plate 27 may move downwards under an action of the first spring 21, so as to drive the first stop block 34 to move downwards. The first stop block 34, when descending, may make contact with the corresponding step surface 331 of the arc-shaped descending control part 33, due to different heights of the step surfaces 331 on the arc-shaped descending control part 33, the first stop block 34 may stop descending after making contact with the corresponding step surface 331, so as to drive the treatment needle 7 to descend to a corresponding depth, thus demands for different acupuncture depths are met, and the reset tube may be reset through the reset rod.

As shown in FIG. 9, for example, the placebo needle kit 2 has a shape consistent with that of the treatment needle kit 1. The placebo needle kit 2 includes a second needle tube 35, a second descending component 36, a second depth adjustment component 37, a second descending control component 38 and a placebo needle 39.

The second depth adjustment component 37, the second descending component 36 and the placebo needle 39 are arranged in the second needle tube 35 in sequence from top to bottom. The second descending control component 38 is arranged in the second needle tube 35 and located below the second descending component 36.

The second depth adjustment component 37 is connected with the second descending component 36, the placebo needle 39 is in sliding connection with the second descending component 36, the second descending control component 38 is arranged in the second needle tube 35 and located below the second descending component 36, the second descending control component 38 is configured to adjust a position of the second descending component 36 in the second needle tube 35, and the second descending component 36 is configured to control a descending distance of the placebo needle 39 to be a set value when moving downwards in the second needle tube 35. Generally, the descending distance of the placebo needle 39 is in a range from 1 mm to 3 mm, that is, only a needle hole is left in the skin of a patient.

For example, the second needle tube 35 has the same structure as the first needle tube 3, and the second descending control component 38 has the same structure as the first descending control component 6, which is omitted here.

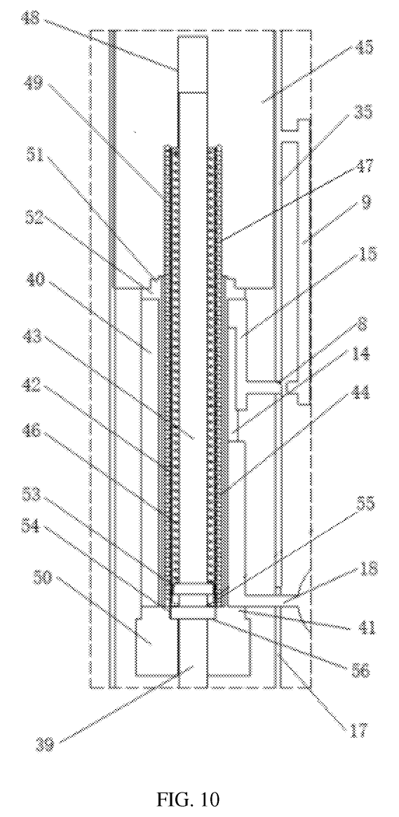

As shown in FIG. 10, for example, the second descending component 36 includes a second reset tube 40, a second connecting cylinder 41, an inner spring sleeve 42, a first acupuncture fixing rod 43 and a second spring 44. The second reset tube 40 has the same structure as the first reset tube 11, and the second connecting cylinder 41 has the same structure as the first connecting cylinder 12, which is omitted here.

For example, the second depth adjustment component 37 includes a second color recognizing part and a third spring 46. The second color recognizing part is arranged at a top opening of the second needle tube 35 and includes a second color recognizing body 45 and a second spring sleeve 47, the second color recognizing body 45 is cylindrical, a bottom of the second color recognizing body 45 is rotationally connected with the top opening of the second needle tube 35, and a top of the second color recognizing body 45 is located outside the second needle tube 35, so the user may rotate the second color recognizing body 45.

A plurality of color regions are arranged in a position, having the same height as the color recognizing port 19 of the second needle tube 35, of an outer side surface of the second color recognizing body 45 in a circumferential direction, and colors between the plurality of color regions are different. The plurality of color regions have the same size as the color recognizing port 19 of the second needle tube 35, each color region is of a small blocky protruding structure, the plurality of color regions horizontally surround the second color recognizing body 45 by one circle, and each color region may be caused to rightly fall into the color recognizing port 19 of the second needle tube 35 by rotating the second color recognizing body 45.

A first round hole 48 and a second round hole 49 which communicate with each other are formed in a middle of the second color recognizing body 45 in sequence from top to bottom in an axial direction, and an inner diameter of the second round hole 49 is greater than an inner diameter of the first round hole 48.

A top of the second spring sleeve 47 is in interference fit with the second round hole 49, and a disc-shaped second base plate 50 is fixedly arranged below the limiting ring 13 at a bottom of the second connecting cylinder 41.

The bottom of the second color recognizing body 45 is in transmission connection with a top of the second connecting cylinder 41. For example, the second depth adjustment component 37 further includes a second internal gear 51, the second descending component 36 further includes a second external gear 52, the second internal gear 51 is arranged on an outer side of the second spring sleeve 47 in a sleeving mode and located at the bottom of the second color recognizing body 45, and the second external gear 52 engaged with the second internal gear 51 is arranged at the top of the second connecting cylinder 41.

A bottom of the second spring sleeve 47 is located in the second connecting cylinder 41, the second spring 44 is arranged in the second spring sleeve 47, a top of the second spring 44 is in contact with a top of the second round hole 49, and a bottom of the second spring 44 is in contact with the second base plate 50.

The inner spring sleeve 42 is arranged in the second spring sleeve 47, a top of the inner spring sleeve 42 is fixedly connected with the top of the second round hole 49, the second spring 44 is arranged outside the inner spring sleeve 42 in a sleeving mode, the first acupuncture fixing rod 43 is located in the inner spring sleeve 42, a top of the first acupuncture fixing rod 43 is in sliding connection with the first round hole 48, and a bottom of the first acupuncture fixing rod 43 penetrates through the second base plate 50 to be fixedly connected with the placebo needle 39.

The first acupuncture fixing rod 43 is provided with a first stop ring 53 and a second stop ring 54, the first stop ring 53 is located in the inner spring sleeve 42, the second stop ring 54 is located below the inner spring sleeve 42, a first inner flange 55 is arranged at a bottom of the inner spring sleeve 42 and located between the first stop ring 53 and the second stop ring 54, a second groove 56 matching the second stop ring 54 is formed in a connection position of the second base plate 50 and the second connecting cylinder 41, the third spring 46 is arranged on the first acupuncture fixing rod 43 in a sleeving mode, a top of the third spring 46 is in contact with the top of the second round hole 49, and a bottom of the third spring 46 is in contact with the first stop ring 53.

In an idle state, the second base plate 50 pushes the second stop ring 54 upwards, so that the third spring 46 is in a compressed state. When the second base plate 50 descends, the third spring 46 is released to push the first stop ring 53 downwards, the first acupuncture fixing rod 43 slides in the first round hole 48, and the first stop ring 53 does not descend when descending to make contact with the first inner flange 55 of the inner spring sleeve 42. Thus, no matter how to rotate each color region of the second color recognizing body 45, moving distances of the placebo needle 39 located on the first acupuncture fixing rod 43 are the same.

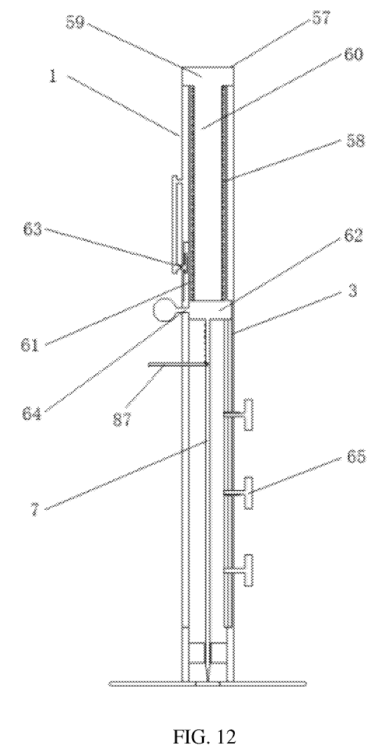

As shown in FIG. 12, for example, the first depth adjustment component 5 includes a first plunger 57 and a fourth spring 58. The first plunger 57 is in a “T” shape and includes a third cylindrical portion 59 and a fourth cylindrical portion 60 which are connected in sequence from top to bottom, an outer diameter of the third cylindrical portion 59 is greater than an outer diameter of the fourth cylindrical portion 60, the third cylindrical portion 59 is rotationally connected with the top opening of the first needle tube 3, the fourth cylindrical portion 60 is located in the first needle tube 3, a second cavity is formed between the fourth cylindrical portion 60 and the first needle tube 3, and the fourth spring 58 is located in the second cavity and arranged on the fourth cylindrical portion 60 in a sleeving mode.

As shown in FIG. 12, for example, the first descending component 4 includes a first descending tube 61. The first descending tube 61 is arranged on an outer side of the fourth spring 58 in a sleeving mode and located between the first needle tube 3 and the fourth spring 58, a second stop block 62 is arranged at a bottom of the first descending tube 61, and a bottom of the second stop block 62 is fixedly connected with the treatment needle 7.

A second buckle 63 matching the snap-in opening 8 of the first needle tube 3 is arranged on an outer side surface of the first descending tube 61. The second buckle 63 has the same structure as the first buckle 15.

For example, the first descending component 4 further includes a second reset rod 64, the second reset rod 64 penetrates through a reset groove 17, one end of the second reset rod 64 is fixedly connected with the second stop block 62 at the bottom of the first descending tube 61, the other end of the second reset rod 64 is located outside the first needle tube 3, and the other end of the second reset rod 64 is provided with a spherical handle, so an operation is convenient.

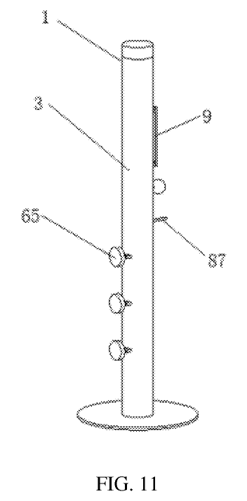

As shown in FIGS. 11 and 12, for example, the first descending control component 6 includes a plurality of descending control buttons 65 which are vertically distributed on the first needle tube 3.

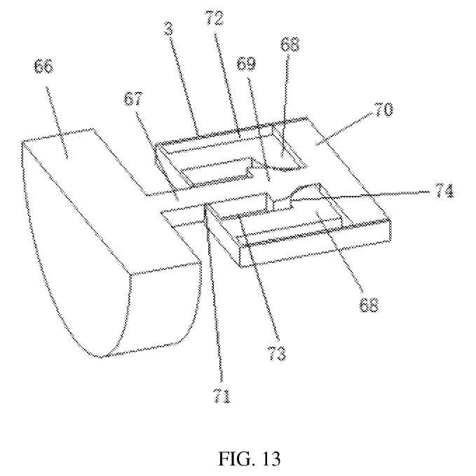

As shown in FIG. 13, each descending control button 65 includes a press plate 66, a motion bar 67, two clamping blocks 68, a locking block 69 and a stop bar 70. A third round hole 71 and a fourth round hole 72 which communicate with each other are formed in sequence in a position, at each descending control button 65, of the tube wall of the first needle tube 3 from outside to inside, and an inner diameter of the fourth round hole 72 is greater than an inner diameter of the third round hole 71.

The two clamping blocks 68 are oppositely arranged in the fourth round hole 72, one ends, close to the third round hole 71, of the two clamping blocks 68 are fixedly connected with a side wall of the fourth round hole 72, and a third cavity is formed between the two clamping blocks 68.

The press plate 66 is arranged outside the first needle tube 3, the hemispherical locking block 69 is arranged in the third cavity, one end of the motion bar 67 is fixedly connected with the press plate 66, the other end of the motion bar 67 penetrates through the third round hole 71 to be fixedly connected with the hemispherical locking block 69, the motion bar 67 is in sliding connection with the third round hole 71, the stop bar 70 is arranged in the fourth round hole 72 and located at one ends, away from the third round hole 71, of the two clamping blocks 68, and the locking block 69 is fixedly connected with the stop bar 70.

For example, the third cavity includes a cylindrical segment 73 and an arc-shaped segment 74 which communicate with each other from outside to inside, and an arc surface of the arc-shaped segment 74 and an arc surface of the locking block 69 are tangential to form clamping for the locking block 69, so that the motion bar 67 and the stop bar 70 are fixed in position.

By pressing the press plate 66, the locking block 69 may be pressed into the arc-shaped segment 74 and form clamping. When in use, the elastic push part 9 is pressed to separate the second buckle 63 from the snap-in opening 8, the first descending tube 61 drives the treatment needle 7 to descend under an action of the fourth spring 58, and the second stop block 62 stops descending when descending to make contact with the stop bar 70 of the set descending control button 65.

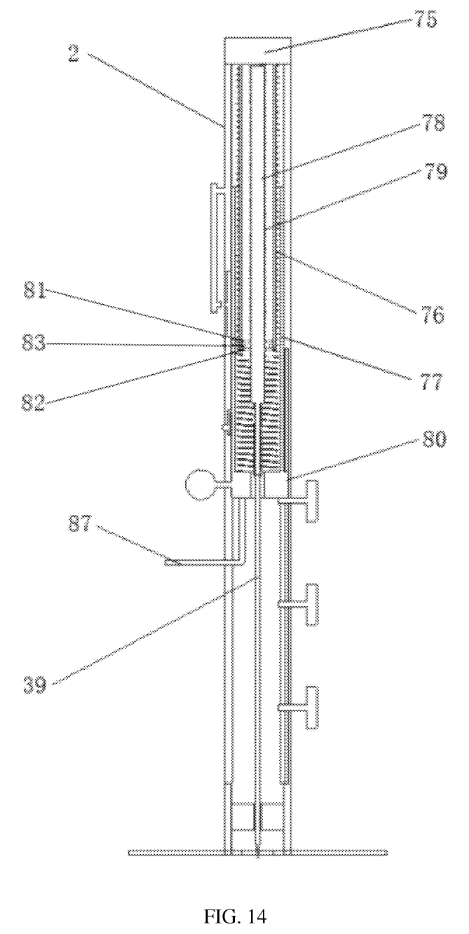

As shown in FIG. 14, for example, the second depth adjustment component 37 includes a second plunger 75 and a fifth spring 76. The second plunger 75 is in a “T” shape and includes a fifth cylindrical portion and a sixth cylindrical portion which are connected in sequence from top to bottom, an outer diameter of the fifth cylindrical portion is greater than an outer diameter of the sixth cylindrical portion, the fifth cylindrical portion is rotationally connected with the top opening of the second needle tube 35, the sixth cylindrical portion is located in the second needle tube 35, a fourth cavity is formed between the sixth cylindrical portion and the second needle tube 35, and the fifth spring 76 is located in the fourth cavity and arranged on the sixth cylindrical portion in a sleeving mode.

The second descending component 36 includes a second descending tube 77, a second acupuncture fixing rod 78 and a sixth spring 79. The second descending tube 77 has the same structure as the first descending tube 61, which is omitted.

The second descending tube 77 is arranged on an outer side of the fifth spring 76 in a sleeving mode and located between the second needle tube 35 and the fifth spring 76, and a third stop block 80 is arranged at a bottom of the second descending tube 77.

A fifth cavity is formed in the second plunger 75 in an axial direction, the second acupuncture fixing rod 78 is located in the fifth cavity, and a bottom of the second acupuncture fixing rod 78 penetrates through the third stop block 80 to be fixedly connected with the placebo needle 39.

The second acupuncture fixing rod 78 is provided with a third stop ring 81 and a fourth stop ring 82, a second inner flange 83 is arranged at a bottom of the second plunger 75 and located between the third stop ring 81 and the fourth stop ring 82, the third stop ring 81 is located in the fifth cavity, the fourth stop ring 82 is located below the fifth cavity, the sixth spring 79 is arranged on the second acupuncture fixing rod 78 in a sleeving mode, a top of the sixth spring 79 is in contact with a top of the fifth cavity, and a bottom of the sixth spring 79 is in contact with the third stop ring 81. When this solution is in use, the placebo needle 39 does not descend with descending of the descending tube.

In the above embodiment, when in use, no matter which acupuncture depth is set, the placebo needle kit 2 can prick the skin only in a depth ranging from 1 mm to 3 mm.

In an acupuncture double-blind clinical study, the treatment needle kit 1 and the placebo needle kit 2 are randomly distributed to different patients. Meanwhile, the patients do not know which specific type of kits they are distributed to. Such random distribution and unknowingness further guarantee effectiveness of the double blind design. The placebo needle kit 2 has the appearance design completely identical with that of the treatment needle kit 1, so that the user cannot judge which needle kit it is according only to the appearance. Operation processes of the two types of kits are also completely the same, and the patients cannot distinguish true and false through the operations. In addition, the patients have the same touch feeling, and needle heads of both the treatment needle kit 1 and the placebo needle kit 2 touch the subjects.

For example, a fixing plate 84 is arranged below bottoms of the first needle tube 3 and the second needle tube 35, and adhesive tape 88 is detachably arranged at a bottom of the fixing plate 84. The kit is fixed to the skin through the adhesive tape 88, so that the kit is prevented from falling or moving during acupuncturing.

For example, a first through hole is formed in the fixing plate 84 at a position of the adhesive tape 88, and a blood-mimicking member 85 is arranged in the first through hole. The blood-mimicking member 85 includes a cotton ball soaked with iodophor, and a plastic film is arranged outside the cotton ball. When in use, both the treatment needle 7 and the placebo needle 39 may prick the cotton ball, so the pricked skin may be subjected to disinfection treatment, and meanwhile, color of the iodophor may cover a difference of a treatment group and a placebo group caused by a difference of a bleeding amount.

For example, sterile red dry cotton balls are arranged on inner sides of the bottoms of the first needle tube 3 and the second needle tube 35, and the dry cotton balls are used for absorbing bloodstains. The bleeding amount is usually small during acupuncturing, and there is no bleeding sometime, so the red dry cotton ball may cover the traces of bleeding even though there is a small bleeding amount.

For example, acupuncture guide blocks 86 are arranged at bottom openings of the first needle tube 3 and the second needle tube 35, the arc-shaped descending control part 33 of the first descending control component 6 surrounds an outer side of the acupuncture guide block 86 in the first needle tube 3, and the arc-shaped descending control part 33 of the second descending control component 38 surrounds an outer side of the acupuncture guide block 86 in the second needle tube 35. A second through hole matching the treatment needle 7 in diameter is formed in the acupuncture guide block 86 in the first needle tube 3, and a second through hole matching the placebo needle 39 in diameter is formed in the acupuncture guide block 86 in the second needle tube 35, so that the treatment needle 7 and the placebo needle 39 can smoothly penetrate through the acupuncture guide blocks 86.

An outer wall of the acupuncture guide block 86 in the first needle tube 3 is fixedly connected with an inner wall of the arc-shaped descending control part 33 of the first descending control component 6, and a height of the acupuncture guide block 86 in the first needle tube 3 is less than a height of a lowest step surface 331 of the arc-shaped descending control part 33 of the first descending control component 6.

The acupuncture guide blocks 86 are used for improving stability of acupuncturing. During acupuncturing, due to softness and elasticity of the human skin, the needle may shake or shift slightly sometime. However, a fixing channel may be provided due to the presence of the acupuncture guide blocks 86, so the needle can smoothly prick the skin along a preset path, and possibility of shifting and shaking is reduced (similar to a pinching needle insertion method, and accidents such as needle bending and breaking are prevented).

For example, the treatment needle kit 1 and the placebo needle kit 2 each include an electroacupuncture joint 87, each electroacupuncture joint 87 is in an L shape, one end of the electroacupuncture joint 87 of the treatment needle kit 1 is located outside the first needle tube 3, and the other end of the electroacupuncture joint of the treatment needle kit penetrates through the first needle tube 3 to be fixedly connected with a bottom of the first descending component 4. For example, the other end of the electroacupuncture joint 87 of the treatment needle kit 1 penetrates through the first needle tube 3 to be fixedly connected with the bottom of the second stop block 62.

One end of the electroacupuncture joint 87 of the placebo needle kit 2 is located outside the second needle tube 35, and the other end of the electroacupuncture joint of the placebo needle kit penetrates through the second needle tube 35 to be fixedly connected with a bottom of the second descending component 36. For example, the other end of the electroacupuncture joint 87 of the placebo needle kit 2 penetrates through the second needle tube 35 to be fixedly connected with the bottom of the third stop block 80.

The electroacupuncture joints 87 are made of a metal material, and both the second stop block 62 and the third stop block 80 are made of metal materials, for in some cases, the treatment needle 7 needs to be powered on to perform electroacupuncture treatment on the patients.

To sum up, the present disclosure effectively overcomes disadvantages in the prior art and thus has a high industrial applicability value.

Though the present disclosure is described in detail with reference to the aforementioned embodiments, those ordinarily skilled in the art should understand that they can still modify the technical solutions recorded in the aforementioned embodiments, or perform equivalent replacement on some technical features; and these modifications or replacements do not make the essence of the corresponding technical solutions depart from the spirit and scope of the technical solutions of the embodiments of the present disclosure.

1. An acupuncture apparatus for a blind method, comprising a treatment needle kit (1), wherein the treatment needle kit (1) comprises a first needle tube (3) and a first descending control component (6) as well as a first depth adjustment component (5), a first descending component (4) and a treatment needle (7) which are arranged in the first needle tube (3) in sequence from top to bottom;
wherein the first depth adjustment component (5) is connected with the first descending component (4), a top of the treatment needle (7) is fixedly connected with the first descending component (4), and the first descending component (4) drives the treatment needle (7) to move downwards in the first needle tube (3); and the first descending control component (6) is arranged in the first needle tube (3) and located below the first descending component (4), and the first descending control component (6) is configured to adjust a position of the first descending component (4) in the first needle tube (3) so as to control a descending distance of the treatment needle (7); and
both a top and a bottom of the first needle tube (3) are open, a snap-in opening (8) which penetrates through a tube wall is formed in a position, at the first descending component (4), of the first needle tube (3), an outer side wall of the first needle tube (3) is provided with an elastic push part (9) at the snap-in opening (8), an upper end of the elastic push part (9) is fixedly connected with the outer side wall of the first needle tube (3), and a first protrusion block (10) matching the snap-in opening (8) is arranged on an inner side of a lower end of the elastic push part (9).

2. The acupuncture apparatus for the blind method according to claim 1, wherein the first descending component (4) comprises a first reset tube (11) and a first connecting cylinder (12);
wherein the first reset tube (11) is arranged in the first needle tube (3) and located below the first depth adjustment component (5), and a first cavity is formed between the first reset tube (11) and the first needle tube (3); the first reset tube (11) is arranged outside the first connecting cylinder (12) in a sleeving mode, an outer wall of the first connecting cylinder (12) is provided with limiting rings (13) at two ends of the first reset tube (11), and an outer diameter of each limiting ring (13) is greater than an outer diameter of the first reset tube (11); and
a first groove (14) is formed in a position, close to the snap-in opening (8), of a tube wall of a top of the first reset tube (11), a first buckle (15) is arranged in the first groove (14), the first buckle (15) is in a strip shape, a top of the first buckle (15) is fixedly connected with a wall surface of a top of the first groove (14), a second protrusion block (16) matching the snap-in opening (8) is arranged on an outer side surface of a bottom of the first buckle (15), the second protrusion block (16), when unstressed, penetrates through the first cavity to be snapped into the snap-in opening (8), and the second protrusion block (16) slides in the first cavity.

3. The acupuncture apparatus for the blind method according to claim 2, wherein a strip-shaped reset groove (17) is formed in a lower portion of a tube wall of the first needle tube (3), the reset groove (17) extends in an axial direction of the first needle tube (3), a first reset rod (18) is arranged on the first reset tube (11), the first reset rod (18) penetrates through the first cavity and the reset groove (17), one end of the first reset rod (18) is fixedly connected with an outer side wall of a bottom of the first reset tube (11), the other end of the first reset rod (18) is located outside the first needle tube (3), and the other end of the first reset rod (18) is provided with a spherical handle.

4. The acupuncture apparatus for the blind method according to claim 3, wherein the first needle tube (3) is provided with a color recognizing port (19) in an upper portion of the elastic push part (9), and the first depth adjustment component (5) comprises a first color recognizing part (20) and a first spring (21);
wherein the first color recognizing part (20) is arranged at a top opening of the first needle tube (3), the first color recognizing part (20) comprises a first color recognizing body (22) and a first spring sleeve (23), a bottom of the first color recognizing body (22) is rotationally connected with the top opening of the first needle tube (3), and a top of the first color recognizing body (22) is located outside the first needle tube (3); and a plurality of color regions are arranged in a position, having the same height as the color recognizing port (19) of the first needle tube (3), of an outer side surface of the first color recognizing body (22) in a circumferential direction, and colors between the plurality of color regions are different.

5. The acupuncture apparatus for the blind method according to claim 4, wherein the first depth adjustment component (5) further comprises a first internal gear (24), and the first descending component (4) further comprises a first external gear (25);
a blind hole (26) is formed in a middle of the bottom of the first color recognizing body (22) in an axial direction, a top of the first spring sleeve (23) is in interference fit with the blind hole (26), the first internal gear (24) is arranged on an outer side of the first spring sleeve (23) in a sleeving mode and located at the bottom of the first color recognizing body (22), a top of the first connecting cylinder (12) is provided with the first external gear (25) engaged with the first internal gear (24), a first base plate (27) is fixedly arranged below the limiting ring (13) at a bottom of the first connecting cylinder (12), and the top of the treatment needle (7) is fixedly connected with a bottom of the first base plate (27); and
a bottom of the first spring sleeve (23) is located in the first connecting cylinder (12), the first spring (21) is arranged in the first spring sleeve (23), a top of the first spring (21) is in contact with a bottom of the blind hole (26), and a bottom of the first spring (21) is in contact with the first base plate (27).

6. The acupuncture apparatus for the blind method according to claim 5, wherein the first descending control component (6) comprises an arc-shaped descending control part (33), an outer wall surface of the arc-shaped descending control part (33) is attached to an inner wall surface of the first needle tube (3), the arc-shaped descending control part (33) is provided with a plurality of step surfaces (331) having different heights in a circumferential direction, a position of each step surface (331) corresponds to a position of one color region on the first color recognizing part (20), an arc-shaped first stop block (34) is arranged on an outer side surface of the first base plate (27), an arc length of a cross section of the first stop block (34) is equal to an arc length of each step surface (331), and each step surface (331) completely coincides with the first stop block (34) in a vertical direction.

7. The acupuncture apparatus for the blind method according to claim 1, wherein the first depth adjustment component (5) comprises a first plunger (57) and a fourth spring (58);
wherein the first plunger (57) is in a “T” shape and comprises a third cylindrical portion (59) and a fourth cylindrical portion (60) which are connected in sequence from top to bottom, an outer diameter of the third cylindrical portion (59) is greater than an outer diameter of the fourth cylindrical portion (60), the third cylindrical portion (59) is rotationally connected with a top opening of the first needle tube (3), the fourth cylindrical portion (60) is located in the first needle tube (3), a second cavity is formed between the fourth cylindrical portion (60) and the first needle tube (3), and the fourth spring (58) is located in the second cavity and arranged on the fourth cylindrical portion (60) in a sleeving mode.

8. The acupuncture apparatus for the blind method according to claim 7, wherein the first descending component (4) comprises a first descending tube (61);
wherein the first descending tube (61) is arranged on an outer side of the fourth spring (58) in a sleeving mode and located between the first needle tube (3) and the fourth spring (58), a second stop block (62) is arranged at a bottom of the first descending tube (61), and a bottom of the second stop block (62) is fixedly connected with the treatment needle (7); and a second buckle (63) matching the snap-in opening (8) of the first needle tube (3) is arranged on an outer side surface of the first descending tube (61).

9. The acupuncture apparatus for the blind method according to claim 8, wherein the first descending component (4) further comprises a second reset rod (64), the second reset rod (64) penetrates through a reset groove (17) at a lower portion of a tube wall of the first needle tube (3), one end of the second reset rod (64) is fixedly connected with the second stop block (62), the other end of the second reset rod (64) is located outside the first needle tube (3), and the other end of the second reset rod (64) is provided with a spherical handle.

10. The acupuncture apparatus for the blind method according to claim 1, wherein the first descending control component (6) comprises a plurality of descending control buttons (65) which are vertically distributed on the first needle tube (3);
wherein each descending control button (65) comprises a press plate (66), a motion bar (67), two clamping blocks (68), a locking block (69) and a stop bar (70), a third round hole (71) and a fourth round hole (72) which communicate with each other are formed in sequence in a position, at each descending control button (65), of a tube wall of the first needle tube (3) from outside to inside, and an inner diameter of the fourth round hole (72) is greater than an inner diameter of the third round hole (71);
the two clamping blocks (68) are oppositely arranged in the fourth round hole (72), one ends, close to the third round hole (71), of the two clamping blocks (68) are fixedly connected with a side wall of the fourth round hole (72), and a third cavity is formed between the two clamping blocks (68);
the press plate (66) is arranged outside the first needle tube (3), the hemispherical locking block (69) is arranged in the third cavity, one end of the motion bar (67) is fixedly connected with the press plate (66), the other end of the motion bar (67) penetrates through the third round hole (71) to be fixedly connected with the locking block (69), the motion bar (67) is in sliding connection with the third round hole (71), the stop bar (70) is arranged in the fourth round hole (72) and located at one ends, away from the third round hole (71), of the two clamping blocks (68), and the locking block (69) is fixedly connected with the stop bar (70); and
the third cavity comprises a cylindrical segment (73) and an arc-shaped segment (74) which communicate with each other from outside to inside, and an arc surface of the arc-shaped segment (74) and an arc surface of the locking block (69) are tangential to form clamping for the locking block (69).

11. The acupuncture apparatus for the blind method according to claim 1, further comprising a placebo needle kit (2) having the consistent shape as the treatment needle kit (1), wherein the placebo needle kit (2) comprises a second needle tube (35) and a second descending control component (38) as well as a second depth adjustment component (37), a second descending component (36) and a placebo needle (39) which are arranged in the second needle tube (35) in sequence from top to bottom;
wherein the second depth adjustment component (37) is connected with the second descending component (36), the placebo needle (39) is in sliding connection with the second descending component (36), the second descending control component (38) is arranged in the second needle tube (35) and located below the second descending component (36), the second descending control component (38) is configured to adjust a position of the second descending component (36) in the second needle tube (35), and the second descending component (36) is configured to control a descending distance of the placebo needle (39) to be a set value when moving downwards in the second needle tube (35).

12. The acupuncture apparatus for the blind method according to claim 11, wherein the second depth adjustment component (37) comprises a second color recognizing part and a third spring (46);
the second color recognizing part is arranged at a top opening of the second needle tube (35) and comprises a second color recognizing body (45) and a second spring sleeve (47), a bottom of the second color recognizing body (45) is rotationally connected with the top opening of the second needle tube (35), and a top of the second color recognizing body (45) is located outside the second needle tube (35);
a first round hole (48) and a second round hole (49) which communicate with each other are formed in a middle of the second color recognizing body (45) in sequence from top to bottom in an axial direction, and an inner diameter of the second round hole (49) is greater than an inner diameter of the first round hole (48); and a top of the second spring sleeve (47) is in interference fit with the second round hole (49).

13. The acupuncture apparatus for the blind method according to claim 12, wherein the second descending component (36) comprises a second reset tube (40), a second connecting cylinder (41), an inner spring sleeve (42), a first acupuncture fixing rod (43) and a second spring (44);
wherein a second base plate (50) is fixedly arranged below the limiting ring (13) at a bottom of the second connecting cylinder (41), and the bottom of the second color recognizing body (45) is in transmission connection with a top of the second connecting cylinder (41);
a bottom of the second spring sleeve (47) is located in the second connecting cylinder (41), a top of the inner spring sleeve (42) is fixedly connected with a bottom of the second round hole (49), the second spring (44) is arranged outside the inner spring sleeve (42) in a sleeving mode, a top of the second spring (44) is in contact with the bottom of the second round hole (49), and a bottom of the second spring (44) is in contact with the second base plate (50);
the first acupuncture fixing rod (43) is located in the inner spring sleeve (42), a top of the first acupuncture fixing rod (43) is in sliding connection with the first round hole (48), and a bottom of the first acupuncture fixing rod (43) penetrates through the second base plate (50) to be fixedly connected with the placebo needle (39); and
the first acupuncture fixing rod (43) is provided with a first stop ring (53) and a second stop ring (54), the first stop ring (53) is located in the inner spring sleeve (42), the second stop ring (54) is located below the inner spring sleeve (42), a first inner flange (55) is arranged at a bottom of the inner spring sleeve (42) and located between the first stop ring (53) and the second stop ring (54), a second groove (56) matching the second stop ring (54) is formed in a connection position of the second base plate (50) and the second connecting cylinder (41), the third spring (46) is arranged on the first acupuncture fixing rod (43) in a sleeving mode, a top of the third spring (46) is in contact with a top of the second round hole (49), and a bottom of the third spring (46) is in contact with the first stop ring (53).

14. The acupuncture apparatus for the blind method according to claim 13, wherein the second depth adjustment component (37) comprises a second plunger (75) and a fifth spring (76);
wherein the second plunger (75) is in a “T” shape and comprises a fifth cylindrical portion and a sixth cylindrical portion which are connected in sequence from top to bottom, an outer diameter of the fifth cylindrical portion is greater than an outer diameter of the sixth cylindrical portion, the fifth cylindrical portion is rotationally connected with the top opening of the second needle tube (35), the sixth cylindrical portion is located in the second needle tube (35), a fourth cavity is formed between the sixth cylindrical portion and the second needle tube (35), and the fifth spring (76) is located in the fourth cavity and arranged on the sixth cylindrical portion in a sleeving mode.

15. The acupuncture apparatus for the blind method according to claim 14, wherein the second descending component (36) comprises a second descending tube (77), a second acupuncture fixing rod (78) and a sixth spring (79);
wherein the second descending tube (77) is arranged on an outer side of the fifth spring (76) in a sleeving mode and located between the second needle tube (35) and the fifth spring (76), and a third stop block (80) is arranged at a bottom of the second descending tube (77);
a fifth cavity is formed in the second plunger (75) in an axial direction, the second acupuncture fixing rod (78) is located in the fifth cavity, and a bottom of the second acupuncture fixing rod (78) penetrates through the third stop block (80) to be fixedly connected with the placebo needle (39); and
the second acupuncture fixing rod (78) is provided with a third stop ring (81) and a fourth stop ring (82), a second inner flange (83) is arranged at a bottom of the second plunger (75) and located between the third stop ring (81) and the fourth stop ring (82), the third stop ring (81) is located in the fifth cavity, the fourth stop ring (82) is located below the fifth cavity, the sixth spring (79) is arranged on the second acupuncture fixing rod (78) in a sleeving mode, a top of the sixth spring (79) is in contact with a top of the fifth cavity, and a bottom of the sixth spring (79) is in contact with the third stop ring (81).

16. The acupuncture apparatus for the blind method according to claim 11, wherein a fixing plate (84) is arranged below bottoms of the first needle tube (3) and the second needle tube (35), and adhesive tape (88) is detachably arranged at a bottom of the fixing plate (84).

17. The acupuncture apparatus for the blind method according to claim 16, wherein a first through hole is formed in the fixing plate (84) at a position of the adhesive tape (88), and a blood-mimicking member (85) is arranged in the first through hole.

18. The acupuncture apparatus for the blind method according to claim 11, wherein acupuncture guide blocks (86) are arranged at bottom openings of the first needle tube (3) and the second needle tube (35), the first descending control component (6) surrounds an outer side of the acupuncture guide block (86) in the first needle tube (3), and the second descending control component (38) surrounds an outer side of the acupuncture guide block (86) in the second needle tube (35); and
a second through hole matching the treatment needle (7) in diameter is formed in the acupuncture guide block (86) in the first needle tube (3), and a second through hole matching the placebo needle (39) in diameter is formed in the acupuncture guide block (86) in the second needle tube (35).

19. The acupuncture apparatus for the blind method according to claim 11, wherein the treatment needle kit (1) and the placebo needle kit (2) each comprise an electroacupuncture joint (87), each electroacupuncture joint (87) is in an L shape, one end of the electroacupuncture joint (87) of the treatment needle kit (1) is located outside the first needle tube (3), and the other end of the electroacupuncture joint of the treatment needle kit penetrates through the first needle tube (3) to be fixedly connected with a bottom of the first descending component (4); and
one end of the electroacupuncture joint (87) of the placebo needle kit (2) is located outside the second needle tube (35), and the other end of the electroacupuncture joint of the placebo needle kit penetrates through the second needle tube (35) to be fixedly connected with a bottom of the second descending component (36).
